(19) United States
(12) Patent Application Publication
(10) Pub. No.: US 2009/0209940 A1
(43) Pub. Date: Aug. 20, 2009

(54) FUSION MANUFACTURE OF MULTI-LUMEN CATHETERS

(75) Inventors: Shekhar D. Nimkar, Swampscott, MA (US); Eric Tobin, North Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: SPIRE CORPORATION, Bedford, MA (US)

(21) Appl. No.: 12/244,544

(22) Filed: Oct. 2, 2008

Related U.S. Application Data
(60) Provisional application No. 61/029,023, filed on Feb. 15, 2008.

(51) Int. Cl. A61M 25/00; B23P 11/00; B32B 37/00

(52) U.S. Cl. 604/523; 29/428; 156/296

(57) ABSTRACT

Methods of forming catheters are disclosed, together with methods of forming split tip catheters. In one aspect of the invention, the manufacturing methods can include the steps of: providing first and second catheter tubes each having a substantially D-shaped cross-section, and attaching at least a portion of longitudinal lengths of the first and second catheter tubes along flat surfaces of the first and second catheter tubes to form a dual lumen catheter assembly. The tubes can be fused along at least about 10%, preferably along at least about 50%, more preferably in some applications along at least about 70%, 80% or 90% of the longitudinal length.

### CROSS REFERENCE TO RELATED APPLICATIONS

The present application claims the priority of U.S. Provisional Application Ser. No. 61/029,023 filed Feb. 15, 2008 entitled “Fusion Manufacture of Multi-Lumen Catheters,” which is herein incorporated by reference in its entirety. This application is also related to commonly owned U.S. patent application Ser. No. ______ filed concurrently herewith entitled “Catheters With Enlarged Arterial Lumens” (Attorney Docket No. 101430-238), U.S. patent application Ser. No. ______ filed concurrently herewith entitled “Fusion Manufacture of Split-Tip Catheters” (Attorney Docket No. 101430-234), and U.S. patent application Ser. No. ______ filed concurrently herewith entitled “Manufacture of Fixed Tip Catheters” (Attorney Docket No. 101430-240), each of which are herein incorporated by reference in their entireties.

### BACKGROUND

The present invention generally relates to catheters and preferably to multi-lumen catheters used for vascular access.

Multi-lumen catheters and, in particular split-tip catheters, are desirable for various treatment applications such as hemodialysis where fluid extraction and return occur simultaneously. Hemodialysis is the separation of metabolic waste products and water from the blood by filtration. Typically, a hemodialysis unit is connected to a patient's body by a catheter. The catheter's distal end is placed in a blood vessel and its proximal end is connected to a hemodialysis unit.

During hemodialysis, a patient's blood typically flows through a double lumen catheter to the hemodialysis unit which provides filtration and controls the flow of blood. A double lumen catheter has two lumens that independently allow fluid extraction and return. For example, one lumen can be used for removing blood from a patient for processing in the hemodialysis machine and the other lumen can be used for subsequently returning the processed blood back to the patient's circulatory system. Such catheters can also include additional lumens for flushing, administration of anticoagulants or the like.

Parameters that can be varied to achieve adequate hemodialysis include blood flow rate, dialysis solution flow rate, and dialyzer competency. Generally, raising the blood flow rate increases dialysis efficiency. However, conditions such as access recirculation decrease efficiency. Access recirculation is the recirculation of treated blood back into the hemodialysis unit. Excess recirculation effectively reduces dialysis efficiency and lengthens the duration of the treatment needed for adequate dialysis. Access recirculation can be particularly of concern when using a double lumen catheter due to the close proximity of the intake and outflow ports at the distal tip of the catheter.

Various double lumen catheter designs have been suggested for the purpose of reducing access recirculation. The distal ends of intake and outflow lumens have been longitudinally spaced 20-30 mm apart to prevent recirculation. For example, Twardowski et al. U.S. Pat. No. 5,569,182 discloses that the lumen for return of blood back into the vein should terminate beyond the extraction lumen. The purpose of this is to prevent cleansed blood, exiting from the outlet point of the catheter, from re-entering the catheter's blood inlet point and returning to the dialysis machine. However, certain disadvantages have been noted by such large longitudinal spacing between the distal ends of the respective lumens. For example, blood flow stagnation in the region of the blood vessel between two widely separated tips can lead to clot formation.

In addition to longitudinal spacing of the distal openings of the lumens, others have suggested that the distal end of a multi-lumen catheter can be split such that the distal tip segments can independently move in the blood vessel to optimize the fluid dynamics of the different functions (blood extraction and blood return). The introduction of an angle between the extraction and return lumens of a split tip catheter can further reduce the likelihood of access recirculation due to greater separation between inflow and outflow lumens.

While various techniques are known for manufacturing catheters, there exists a need for more efficient techniques.

### SUMMARY OF THE INVENTION

Methods of forming catheters are disclosed, together with methods of forming split tip catheters. In one aspect of the invention, the manufacturing methods can include the steps of: providing first and second catheter tubes each having a substantially D-shaped cross-section, and attaching at least a portion of longitudinal lengths of the first and second catheter tubes along flat surfaces of the first and second catheter tubes to form a dual lumen catheter assembly. The tubes can be fused along at least about 10%, preferably along at least about 50%, more preferably in some applications along at least about 70%, 80% or 90% of the longitudinal lengths.

In another aspect, following (or during) formation of the catheter, a non-fused portion of the longitudinal lengths can be secured together with a bioresorbable adhesive to simplify vascular insertion. Following insertion, the tip segments can separate upon dissolution of the adhesive, e.g., over a period of time ranging from 1 second to several days, more preferably from about 1 minute to about one hour, or 5 hours or 10 hours.

The distal portions of the first and second catheter tubes can be oriented in a variety of ways. For example, the distal portions can be separate and diverge from each other at an angle. For another example, the distal portions can be substantially parallel to each other while, in some embodiments, being separate from each other.

The longitudinal lengths of the first and second catheter tubes can be attached together by various techniques. For example, the first and second tubes can be attached by heat bonding or adhesive or chemical reaction bonding.

In one embodiment of the invention, the first and second catheter tubes can be oriented such that one tube extends longitudinally beyond the other tube. For example, a portion of the assembly can be removed to form a first lumen tip segment such that the first catheter tube extends longitudinally beyond the second catheter tube. For another example, two tubes of different longitudinal lengths can be fused together such that the first catheter tube extends longitudinally beyond the second catheter tube. In some embodiments, a second lumen tip segment can be joined to the second catheter tube in fluid communication with the second catheter tube. The second lumen tip segment can have a substantially D-shaped cross-section and/or a cross-section shape different from the second catheter tube.

In some embodiments, the method can include encasing the assembly to smoothen any irregularities along the attached portion of the longitudinal lengths. In another aspect, fluid passage holes can be formed in a side of a distal portion of at least one of the catheter tubes.

In another aspect of the invention, a method of forming a catheter is disclosed including the steps of: providing first and second catheter tubes each having a cross-section including at least one substantially flat-sided surface, and attaching at least a portion of the substantially flat-sided surfaces together to form a catheter assembly. In some embodiments, the method can also include encasing the catheter assembly to smoothen any irregularities along the attached surfaces.

The portions of the first and second catheter tubes can be attached together by various techniques. For example, the substantially flat-sided surfaces of the first and second tubes can be attached by heat bonding or adhesive or chemical reaction bonding.

The method can further include allowing a distal portion of the first catheter tube to extend beyond a distal portion of the second catheter tube when at least a portion of their substantially flat-sided surfaces are attached. Furthermore, a lumen tip segment can be joined to the second catheter tube such that the lumen tip segment is in communication with the second catheter tube.

In still another aspect of the invention a method of forming a split tip catheter is disclosed including the steps of: attaching two tubes together along a portion of substantially flat surfaces of respective longitudinal lengths of the tubes (e.g., along substantially planar edges of respective D-shaped cross-sections of the tubes), and allowing distal portions of each of the tubes to remain unattached from each other. The proximal portions of the tubes can optionally remain unattached from each other.

In some embodiments, the tubes can be fused along at least about 10%, preferably along at least about 50%, more preferably in some applications along at least about 70%, 80% or 90% of the longitudinal lengths. Moreover, a non-attached portion of the longitudinal lengths can be secured together with a bioresorbable adhesive.

In one embodiment of the invention, the tubes can be oriented such that the distal portion of one tube extends longitudinally beyond the distal portion of the other tube. For example, at least part of the distal portion of one of the tubes can be removed to form a first lumen tip segment such that the first lumen tip segment extends longitudinally beyond the other tube. For another example, two tubes of different longitudinal lengths can be fused together such that the distal portion of one tube extends longitudinally beyond the distal portion of the other tube. Additionally, in some embodiments, a lumen tip segment can be joined to the tube with a shorter distal portion such that the lumen tip segment is in communication with the tube with a shorter distal portion.

In certain embodiments, it may be preferable that a catheter tube have a different luminal cross-section than a tube to which it is joined to form a catheter assembly. The invention is also applicable to catheter assemblies having three or more tubes.

Other advantages and features will become apparent from the following description and from the claims.

### BRIEF DESCRIPTION OF THE DRAWINGS

The invention will be more fully understood from the following detailed description taken in conjunction with the accompanying drawings, in which:

FIG. 1 is a schematic view of two tubes in an initial, unattached configuration;

FIG. 2 is a schematic view of an embodiment of the present invention showing a multi-lumen split tip catheter;

FIG. 3 is a schematic view of another embodiment of the present invention showing a multi-lumen catheter with split tips at both ends;

FIG. 4 is a schematic view of an embodiment of the present invention showing a multi-lumen catheter having an angled end portion;

FIG. 5 is a schematic view of another embodiment of the present invention showing a multi-lumen catheter with a staggered end portion;

FIG. 6 is a schematic view of an embodiment of the present invention showing a multi-lumen catheter with separable tip portions held together by an adhesive;

FIG. 7 is a schematic view of an embodiment of the present invention showing a catheter including differently shaped lumens;

FIG. 8 is a cross-section view of an embodiment of the present invention showing a catheter construction utilizing opposed D-shaped lumens;

FIG. 9 is a cross-section view of a variation of the embodiment of FIG. 8 showing opposed D-shaped lumens of different cross-sectional areas;

FIG. 10 is a cross-section view of an embodiment of the present invention showing a catheter construction with two individual circular lumens;

FIG. 11 is a cross-section view of an embodiment of the present invention showing an oval-shaped catheter construction;

FIG. 12 is a cross-section view of an embodiment of the present invention showing a catheter construction with three lumens;

FIG. 13 is a cross-section view of a variation of another embodiment of the present invention showing a catheter construction with three lumens;

FIG. 14 is a schematic, partially cutaway, side view of a catheter according to the present invention;

FIG. 15 is a cross-section view of an embodiment of the present invention showing a catheter construction formed from opposed D-shaped lumen bodies inside an outer sheath;

FIG. 16 is a cross-section view of an embodiment of the present invention showing a catheter construction formed from two individual tubes with circular lumens inside an outer sheath;

FIG. 17 is a schematic, perspective view of an embodiment of the present invention showing a catheter assembly including one tube extending beyond another tube;

FIG. 18 is a schematic, perspective view of an embodiment of the present invention showing a lumen tube attached to a catheter;

FIG. 19 is a schematic, perspective view of a variation of an embodiment of the present invention showing a catheter assembly including one tube extending beyond another tube;

FIG. 20 is a schematic, perspective view of a variation of an embodiment of the present invention showing a lumen tube attached to a catheter assembly;

FIG. 21 is a schematic, perspective view of a variation of an embodiment of the present invention showing a lumen tube attached to a catheter assembly;

FIG. 22 is a schematic, perspective view of a variation of an embodiment of the present invention showing a lumen tube attached to a catheter assembly, where the lumen tube is attached to at least a portion of the septum;

FIG. 23 is a schematic, perspective view of a variation of an embodiment of the present invention showing a lumen tube attached to a catheter assembly, where the lumen tube is attached to at least a portion of the septum using an alternative method;

FIG. 24 is a distal cross-sectional view of another embodiment of the present invention showing alternative adhesive disposition;

FIG. 25 is a distal cross-sectional view of yet another adhesive design;

FIG. 26 is a cross-section view of a variation of an embodiment of the present invention showing a catheter assembly including three lumens;

FIG. 27 is a cross-section view of a variation of an embodiment of the present invention showing a lumen tube attached to another catheter assembly; and

FIG. 28 is a schematic side view of a catheter assembly according to the present invention.

### DETAILED DESCRIPTION

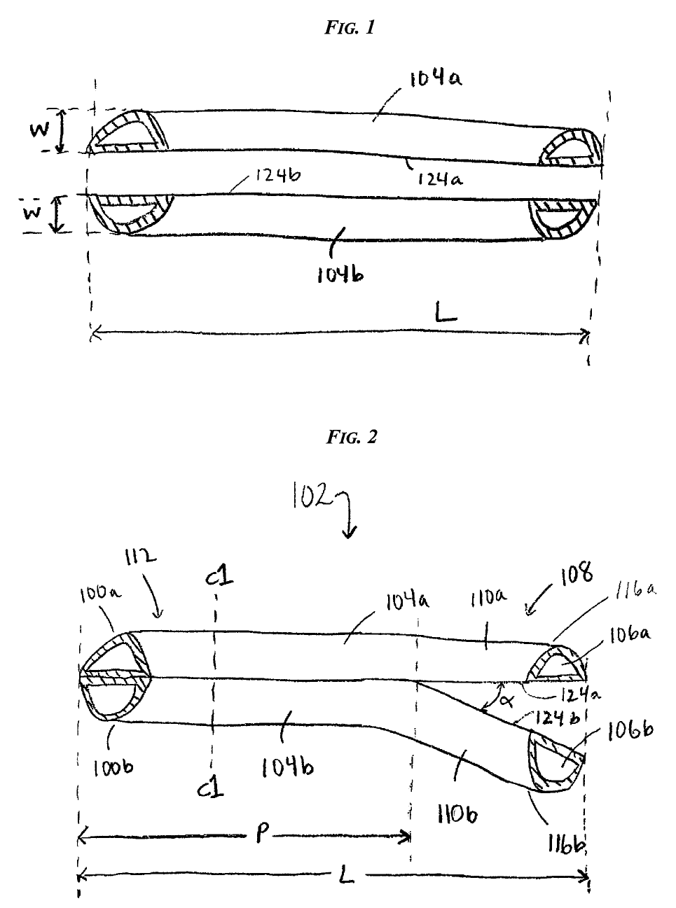

FIG. 1 shows two catheter tubes or bodies 104a, 104b (collectively, the tubes or bodies 104) in an initial, unattached configuration (e.g., prior to their attachment to each other). The tubes 104 include respective inner lumen pathways 106a, 106b (collectively, the lumens or pathways 106) extending longitudinally through the tubes 104. Each of the tubes 104 has a substantially D-shaped cross-section and at least one substantially flat surface (e.g., facing or contacting surfaces 124a, 124b (collectively, the facing or contacting surfaces 124)), although tubes to be attached together can have different cross-sectional shapes. Although the tubes 104 are shown having equal longitudinal lengths L and equal widths W, the tubes 104 can have different longitudinal lengths and/or different widths.

In FIG. 2, an embodiment of a catheter assembly 102 includes the tubes 104 of FIG. 1, which have been attached together. (As used throughout, “the catheter assembly” and its components refers to the various embodiments of the present invention.) The catheter assembly 102 has a fixed tip proximal portion 112 and a split-tip distal portion 108 in which the tubes 104 of the catheter assembly 102 separate into two distal lumen tip segments, 110a, 110b (collectively, the lumen tips 110), although the catheter assembly 102 can have any combination of fixed tips and split tips at its distal and proximal portions 108, 112. The tubes 104 in this embodiment are separate and diverge from one another in the distal portion 108 such that the lumen tip 110b forms an angle α with respect to the other lumen tip 110a. The value of a can be zero or non-zero and is preferably in the range of zero to ninety degrees. One or both distal ends 116a, 116b (collectively, the distal ends 116) and one or both proximal ends 100a, 100b (collectively, the proximal ends 100) of the tubes 104 can be open (as shown in FIG. 2) to provide fluid passageways through the pathways 106, e.g., for blood removal and return. The catheter assembly 102 is typically a very flexible silicone, polyurethane, or other biocompatible composition (e.g., having a stiffness in the range of about 65 to about 85 durometer), and can be fabricated into any type of catheter (e.g., a hemodialysis catheter or a central venous catheter).

The tubes 104 can be made of any biocompatible material, including any material which allows the lumen tips 110 of the tubes 104 to be flexible and facilitate hemodialysis. The distal extraction and return tip portions 110 of each tube 104 include the pathways 106 formed therein for the extraction or return of blood or other bodily fluids. The pathways 106 are preferably sized to allow the carrying of blood to and from a hemodialysis unit, although the pathways 106 can be any size, and the catheter assembly 102 can be used in any application. The lumen tips 110 can be the same length or have different lengths.

The catheter assembly 102 can be formed by taking the two tubes 104 as individual tubes (e.g., as shown in FIG. 1) and fusing the tubes 104 together along at least a portion of their lengths to form the catheter assembly 102. An outer sheath can be added to at least a portion of the catheter assembly 102, as discussed further below, and/or access ports can be added to the tubes 104 at the proximal portion 112. The access ports can include couplings, such as Luer-locks or the like, to couple the proximal portion 112 to a hemodialysis machine in which blood is circulated and purified.

The tubes 104 can be attached together to form the catheter assembly 102 in a variety of ways. For example, in one embodiment, the tubes 104 can be fused along at least a portion of their longitudinal lengths along substantially flat surfaces such as the contacting surfaces 124 of the tubes 104. Any fusion technique can be used, e.g., thermal fusion where elements to be joined (here, outer surfaces of the tubes 104) are heated along any or all portions of their perimeters or other areas to a desired temperature and fused together by application of a desired force and allowing them to melt/cool together. In another example embodiment, the tubes 104 can be fused together using a bonding technique, e.g., applying a bonding material such as an adhesive to one or more of the elements to be bonded and, if necessary, heating the bonding material to bond it to the elements. In some embodiments, the catheter assembly 102 can be formed using any combination of heat fusion and bonding techniques.

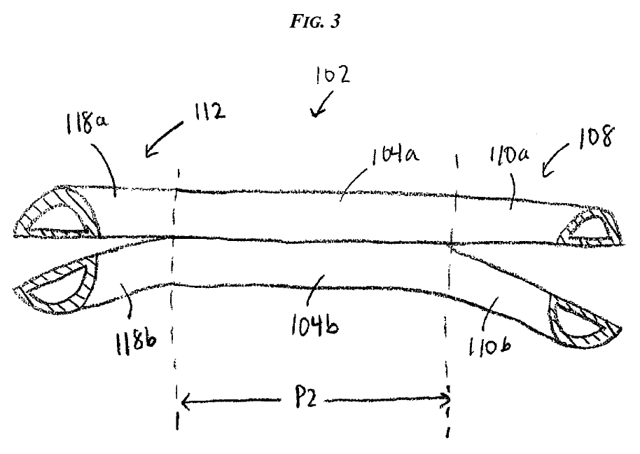

Any portion of each of the tubes 104 can be attached together, e.g., 100% of the longitudinal lengths of one or both tubes 104, about 90% of the longitudinal lengths of one or both tubes 104, etc. If less than 100% of the tubes' longitudinal lengths are attached, the resulting catheter assembly 102 can be used to create a split tip catheter, e.g., by adding one or more additional structures to the catheter assembly 102. As illustrated in FIG. 2, the tubes 104 are fused together along a portion P of their lengths, leaving freely floating, unattached portions (the lumen tips 110) of length L-P at the distal portion 108. In another embodiment, shown in FIG. 3, the tubes 104 can be fused together along a portion P2 of their longitudinal lengths, leaving the lumen tips 110 at the distal portion 108 and leaving similar freely floating, unattached portions (lumen tip segments 118a, 118b (collectively, the lumen tips 118)) at the proximal portion 112.

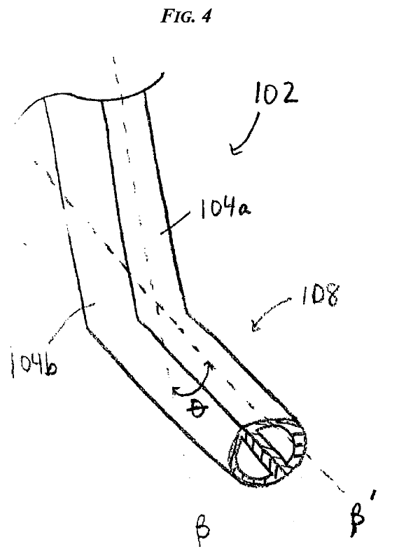

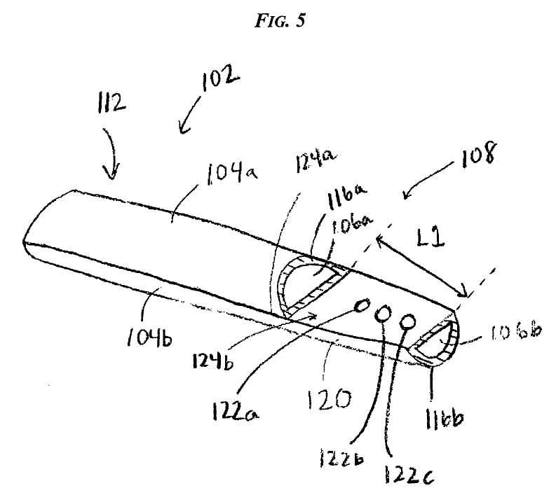

The catheter assembly embodiments illustrated in FIGS. 2-3 show the tubes 104 linearly aligned and substantially parallel to each other along their longitudinal lengths. However, as shown in FIG. 4, the tubes 104 at the distal portion 108 (and/or at the proximal portion 112 (not shown in FIG. 4)) can be substantially parallel to each other in an angled tip configuration, e.g., as described in U.S. Pat. No. 6,482,169, which is hereby incorporated by reference in its entirety. In such a configuration, the distal portion 108, having a distal longitudinal axis β′, is oriented at an angle θ with respect to a longitudinal axis P of the non-angled portion of the catheter assembly 102, where θ can have any value (including zero, such as in the embodiments illustrated in FIGS. 5-7, discussed below). The angle θ can be formed after the tubes 104 have been joined, e.g., by the application of heat. Alternatively, the tubes 104 can have an initial configuration where the distal axis β′ is at the angle θ with respect to the axis β.

The tubes 104 can have different longitudinal lengths, as in yet another embodiment shown in FIG. 5. In FIG. 5, one tube 104b has a longer longitudinal length than the other tube 104a by a length L1. The entire longitudinal length of the shorter tube 104a has been attached to the longer tube 104b, resulting in a freely floating, unattached lumen tip 120 at the distal portion 108 of the longer tube 104b that extends the length L1 beyond the distal end 116a of the shorter tube 104a. The length L1 can be in the range of about 1-3 inches, which is a preferable, but only an example, length of overhanging tube.

The tubes 104 are aligned at the proximal portion 112 in the embodiment shown in FIG. 5, but in other embodiments, one of the tubes 104 could extend any length beyond the other tube at the proximal portion 112, whether or not either of the tubes 104 extends beyond the other at the distal portion 108. Furthermore, whether or not the tubes 104 have equal longitudinal lengths, the catheter assembly 102 can be formed by extending the tubes 104 in a staggered, step configuration such that one of the tubes 104 extends longer than the other tube at the distal portion 108 and/or the proximal portion 112 by any length. By non-limiting example, the tubes 104 can be aligned while hot so at least one of the tubes 104 longitudinally extends beyond the other at the distal and/or proximal portions 108, 112 and can bond together in such a formation as they cool.

Also illustrated in FIG. 5 are fluid passage holes (also called fluid openings) 122a, 122b, 122c (collectively, the fluid passage holes or openings 122) in fluid communication with the pathway 106b of their respective tube 104b to facilitate fluid return (which typically occurs through the longer tube 104b) and/or removal (which typically occurs through the shorter tube 104a), e.g., blood removal and return during hemodialysis. The fluid openings 122 can be of any number, shape, and size and can be located in a variety of places on any of the tubes 104. The fluid openings 122 can be formed in one or more of the tubes 104 prior and/or subsequent to joining the tubes 104. FIG. 5 shows the fluid openings 122 located on the facing surface 124b of the longer tube 104b. Alternatively, or in conjunction with the fluid passage holes 122, one or both of the distal ends 116 of the tubes 104 can be open (as shown in FIG. 5) to provide fluid passageways through the pathways 106. Furthermore, the shorter tube 104a can have distal fluid openings similar to those described for the longer tube 104b, whereby the fluid openings could be exposed, for example, by not fusing the distal portion 108 of the shorter tube 104a to the longer tube 104b or by allowing one or more fluid openings to be exposed upon dissolution of bioresorbable adhesive filling or covering the fluid openings, as described further below.

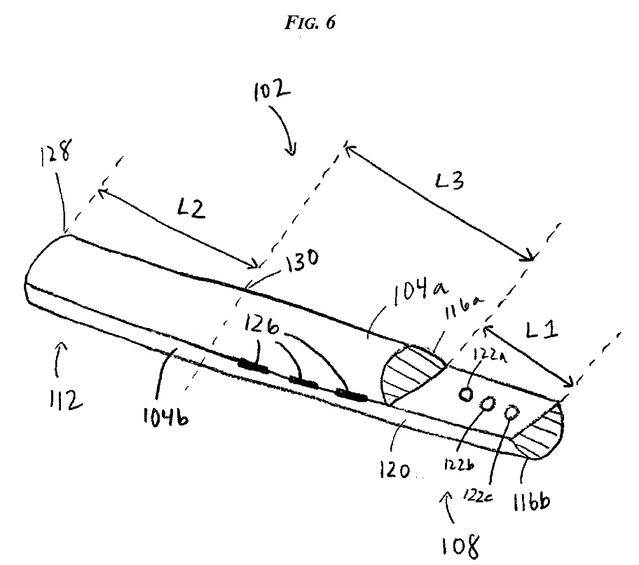

FIG. 6 shows still another embodiment where the tubes 104 have been fused together along a length L2+L3 of their respective longitudinal lengths between a proximal end 128 and a location 130, thereby leaving the freely floating, unattached lumen tip 120 at the distal portion 108 of the longer tube 104b that extends a length L1 beyond the distal end 116a of the shorter tube 104a. Bioresorbable adhesive has been applied to the non-fused length L3 of the facing surfaces 124 of the tubes 104 as discrete spots or regions 126. As used herein, the term “bioresorbable” refers to materials that are biodegradable or biosoluble such that they degrade or break down by mechanical degradation upon interaction with a physiological environment into components that are metabolizable or excretable over a period of time.

The bioresorbable adhesive used to join the tubes 104 to one another can be a composition selected from the group of polymers consisting of polylactides, polyglycolides, polylactones, polyorthoesters, polyanhydrides, and copolymers and combinations thereof. In general, bioresorbable adhesives have bonding elements and degradable elements. The degradable elements can have the components of polylactide, polyglycolide and polylactones (polycaprolactone). The bonding elements can have hydrogen bonding strength (polyvinyl alcohol, polysaccharides) or can be able to polymerize as a single component (cyanoacrylates) or as two components (epoxy compound plus amino compounds, or radical (light) initiators of acrylate compounds).

Proteins, sugars, and starch can also be used as an adhesive. By way of non-limiting example, antithrombotic agents such as heparin and hirudin, citrate, antithrombin-heparin complex, and albumin heparin complex as well as anti-infective agents such as chlorohexidine, silver, antibiotics, and antiseptic agents may be added to the adhesive.

In an embodiment of the present invention, polymers which can be useful include polyurethane, generally described as a copolymer of polyethylene glycol with polylactide or polyglycolide end capped with methacrylates. Another embodiment can include a two component composition, one component preferably including a low molecular weight polyurethane end capped with methacrylates, and the other component preferably including polylactide, polyglycolide, or polycaprolactone end capped with methacrylate.

In another embodiment of the present invention, one or more components can be used from styrene, methyl methacrylate, methyl acrylate, ethylene dimethacrylate, ethylene diacrylate, acrylamide, diurethane dimethacrylate, polyisoprenegraft-maleic acid monomethyl ester, azobis (cyanovaleric acid), azobiscyclohexanecarbonitrile, azobisisobutyronitrile, benzoyl peroxide, iron (II) sulfate, polyvinyl alcohol, dextran, polysaccharide, epichlorohydrin, ethylenediamine, diaminocyclohexane, diamino propane, copolymers with polylactide and polyethylene oxide as the blocks and acrylate, methacrylate as the end groups, cyanoacrylates, ethyl-2cyanoacrylate, propyl-2-cyanoacrylates, pentyl-2-cyanoacrylate, hexyl-2-cyanoacrylate, and octyl-2-cyanoacrylate, ammonium persulfate and/or polyethylene glycol methacrylate when water, organic solvent such as dichloromethane, chloroform, tetrahydrofuran, acetone, petroleum ether, acetyl acetate, dimethylformamide, or the mixture thereof, is combined with the aforementioned solvents.

Additional information on bioresorbable adhesive compositions and catheter assembly manufacturing techniques employing such compositions can be found in commonly-owned, co-pending U.S. patent application Ser. No. 10/874,298 filed Jun. 9, 2004 entitled “Splitable Tip Catheter With Bioresorbable Adhesive”, herein incorporated by reference in it entirety.

The spots 126 of the bioresorbable adhesive can be applied continuously along the entire portion of the longitudinal length of one or both of the tubes 104 or applied selectively in an assortment of areas thereof. Preferably, the bioresorbable adhesive is applied along non-fused portions of both of the facing surfaces 124 such that the spots 126 of adhesive facilitate the joining of the tubes 104 prior to insertion into a blood vessel and allow the distal extraction and return tips of the tubes 104 to separate after insertion. The spots 126 of bioresorbable adhesive can vary in number, size, shape, and distance from one another. In FIG. 6, the spots 126 of adhesive have been applied intermittently along the length L3 of the facing surfaces 124 extending between a location 130 and the distal end 116a of the shorter tube 104a.

In the embodiments described herein, the bioresorbable adhesive preferably dissolves after insertion into a blood vessel to provide separation of the tubes 104 in a time period, e.g., over a period of time ranging from 1 second to several days (or longer), more preferably from about one minute to about ten hours, or five hours or one hour. This time period can be controlled by using different compositions of the bioresorbable adhesive as well as by the amount of adhesive applied to join the tubes 104 together. In an embodiment of the catheter assembly 102 with one or more distal fluid openings 122, the bioresorbable adhesive can be water soluble such that the introduction of saline or similar type fluid will effectuate the separation of the tubes 104 and exposure of the fluid openings 122. In this instance, the bioresorbable adhesive will not dissolve until a time after the introduction of the soluble solution into the tubes 104. Furthermore, the fluid openings 122 can be filled or covered with fluid activated bioresorbable adhesive, whether or not bioresorbable adhesive is otherwise used on the facing surfaces 124 of the tubes 104. After insertion of the catheter assembly 102 into a blood vessel, saline or similar type fluid can be introduced into one or both of the tubes 104 at the open proximal portion 112 such that the fluid travels through the tube(s) 104 to the distal fluid openings 122 and dissolves the fluid activated bioresorbable adhesive, thereby allowing fluid communication between the openings 122 and the lumen pathway(s) 106. In the embodiment shown in FIG. 6, the distal ends 116 of the tubes 104 are closed, and fluid only enters and/or exits the pathways 106 through the openings 122 (and also possibly through the pathways 106 at the proximal portion 112 of the catheter assembly 102). The openings 122 are obscured on the shorter tube 104a until such time one or more of the spots 126 of adhesive dissolve and provide fluid access to one or more of the openings 122. Of course, depending on the lengths of the tubes 104, the openings 122 on both of the tubes 104 could be obscured until such time one or more of the spots 126 dissolve and/or adhesive filling or covering the openings 122 dissolves.

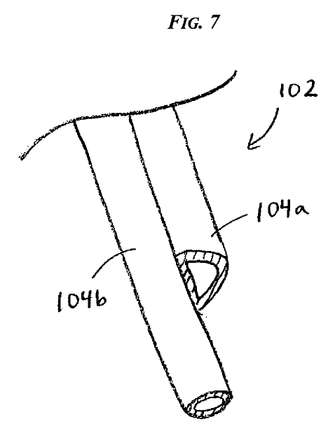

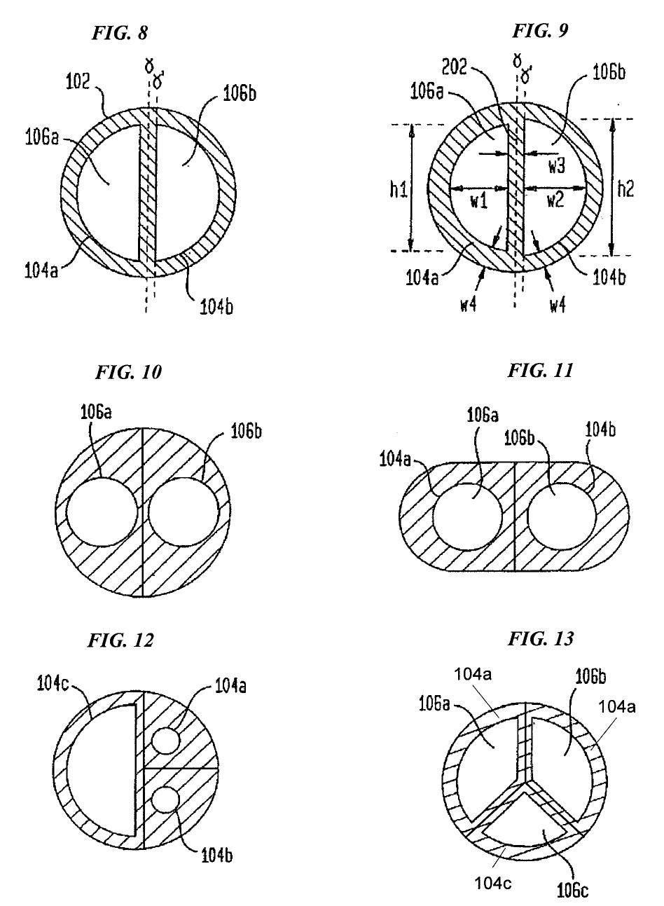

The tubes 104 can have a variety of cross-sectional shapes and sizes but preferably, as shown in the embodiments of FIGS. 1-6, the catheter assembly 102 has a substantially elliptical (circular or oval) shape and the tubes 104 are each substantially D-shaped. However, one or both of the tubes 104 can transition from one shape to another along at least a portion of its length, e.g., transition from a D-shaped cross-section to a circular cross-section. Furthermore, each of the tubes 104 can have a cross-sectional shape, size, or area that can be the same or distinct from the catheter assembly 102 and/or the other tube. One embodiment of the catheter assembly 102 where the tubes 104 have different cross-sectional shapes is shown in FIG. 7, with one tube 104a having a D-shaped cross-section and the other tube 104b having a substantially circular cross-section. A substantially flat-sided surface of the D-shaped tube 104a can be attached to a substantially flat, tangential surface of the substantially circular tube 104b. Examples of c1-c1 cross-sections (see FIG. 2) are illustrated in FIGS. 8-13.

FIG. 8 shows a c1-c1 cross-section view of an embodiment showing a construction utilizing opposed D-shaped tubes 104 having substantially the same size of pathways 106. FIG. 9 is a c1-c1 cross-section view of another embodiment showing opposed D-shaped tubes 104 where one tube 104a is of a smaller size (e.g., smaller cross-sectional area) than the other tube 104b. FIG. 10 is a c1-c1 cross-section view of an embodiment showing an elliptical construction utilizing individual, elliptical lumen pathways 106. FIG. 11 is a c1-c1 cross-section view of another embodiment showing another elliptical construction including two elliptical-shaped pathways 106 in the tubes 104. FIG. 12 is a cross-section view of an embodiment showing three tubes 104, at least one of which (here, tube 104c) having a different size and/or shape from at least one other tube (here, tubes 104a, 104b). FIG. 13 is a cross-section view of a variation of an embodiment showing three tubes 104 having pathways 106 of substantially the same size and shape, although they can have any same or different sizes and shapes.

As mentioned above, an outer sheath, e.g., a fusing tube, can be added to partially or entirely cover and enclose the catheter assembly 102 after the tubes 104 have been joined together. Such an outer sheath can encase the catheter assembly 102 and smoothen any irregularities along the attached portion of the longitudinal lengths of the tubes 104. The outer sheath can be any shape and size and can be made of the same material as the tubes 104 or other material compatible with insertion into a blood vessel. The outer sheath can remain on or be removed from at least a portion of the catheter assembly 102. FIG. 14 illustrates an embodiment of the catheter assembly 102 partially encased by an outer sheath 300 and formed into a split tip catheter 302. As illustrated in this embodiment, the outer sheath 300 terminates proximal to the distal ends 116 of the tubes 104 such that the distal lumen tips 110 of the tubes 104 are separate or can separate from one another after being inserted into a blood vessel. Also shown in FIG. 14 is the proximal portion 112 of the catheter assembly 102 split into the separate lumen tips 118 that terminate with two access ports 132a, 132b.

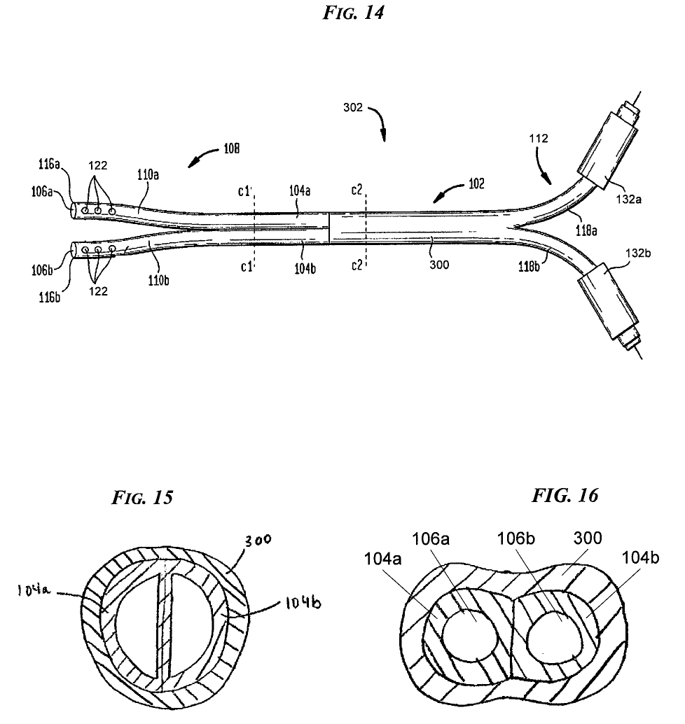

FIG. 15 shows a cross-section c2-c2 (see FIG. 14) of one embodiment of the outer sheath 300. The outer sheath 300 can be of any thickness and can have varying inner and outer shapes as well as varying inner and outer dimensions. The catheter assembly 102 can be constructed such that sheath material 300 encases the tubes 104 and no space remains between the sheath 300 and the tubes 104. For example, the sheath 300 can be fused to the tubes 104 or heat-shrunk around them. FIG. 16 shows another embodiment of the cross-section c2-c2 showing individual, elliptical tubes 104 having substantially circular cross-sectional pathways 106 inside the outer sheath 300.

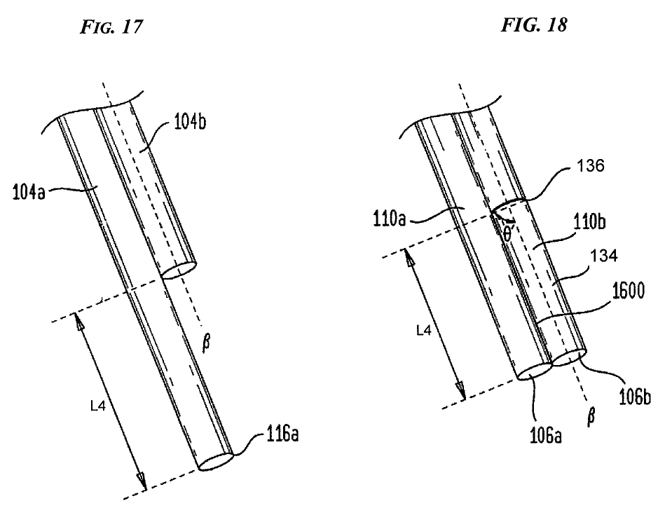

In some embodiments, a lumen tip can be added to one or more of the tubes 104 of the catheter assembly 102, thereby forming a proximal or distal end of a catheter. An exemplary method of forming such a split tip catheter is described with reference to FIGS. 17-26. Although described with reference to these figures (and related ones of FIGS. 1-16), this method (or a similar method) can be implemented to form any of the split tip and/or fixed tip catheter devices described herein.

As shown in FIG. 17, one of the tubes 104b has been attached to the other tube 104a so as to have a longitudinal length less than the other tube 104a by a length L4, where both of the tubes 104 are parallel to each other along the longitudinal axis P. Alternatively, the shorter tube 104b can initially have a longitudinal length as long or longer than the longer tube 104a but subsequently be trimmed, as discussed further below. Once the tubes 104 have desirable longitudinal lengths with respect to one another, a lumen tip segment 134, as shown in FIG. 18, can be joined to the shorter tube 104b at a location 136 such that the shorter tube's tip includes the lumen tip segment 134 and such that the lumen tip segment 134 is in fluid communication with the shorter tube 104b. The tip segment 134 can be similar in size and shape to the tube it is joined to or can be different in size and/or shape. Furthermore, the lumen tip segment 134 can be made from a material different from a material of the shorter tube 104b. The different material can be one more or less flexible than the material of the shorter tube 104b. Using different materials for the lumen tip segment 134 and the shorter tube 104b can allow the catheter assembly 102 to be used more efficiently or to be used at all in an application where it would not be preferable or possible having material of the shorter tube 104b at the distal end 116b. Additionally, the shorter tube 104b can have distal fluid openings similar to those described herein, whereby the fluid openings would typically be included in the lumen tip segment 134 attached to the shorter lumen tip 110b or be subsequently formed in the lumen tip segment 134 after its attachment to the shorter tube 104b.

A tube can be trimmed in a variety of ways. In a preferred example, one of the tubes 104b can be sliced (e.g., cut or scored) widthwise across its circumference at the location 136. Then the length L4 of the cut tube 104b can be trimmed from the catheter assembly 102. In one embodiment according to the invention, with reference to FIG. 8, the end portion of the catheter assembly 102 can be truncated by splitting the assembly along either a center line γ of the longitudinal axis or along an off-center longitudinal axis γ′. In certain applications, truncation along off-center line γ′ can be preferable because it preserves most or all the septum, while sacrificing part of the other tube 104a (e.g., the part extending distally beyond the cut point 136 as shown in FIG. 18).

Referring again to FIG. 9 where one tube 104a is smaller than the other tube 104b, the larger tube 104b is typically the arterial lumen because that is the one of the tubes 104 more prone to clogging in a hemodialysis setting, and a larger size pathway 106b can help reduce clogging. Truncation of the end portion according the invention typically involves sacrificing part of the larger tube 104b and joining a new distal tip segment in its place. The catheter assembly 102 can again be split along an off-center longitudinal axis γ′, thereby preserving most or all of a septum 202, sacrificing part of tube 104b (e.g., the part extending distally beyond the cut point 136). Following truncation, a new distal tip segment 134 can then be joined to the shorter tube 104b of the catheter assembly 102.

In certain applications it can be preferable to sacrifice the smaller tube 104a instead. In such instances, the truncation line can be moved to the other side of the septum 202.

Dimensions of the tubes 104a and 104b can vary between embodiments. In this example embodiment of FIG. 9, dimensions allow the catheter assembly 102 to be used with standard hemodialysis equipment and lumen tip segments. Maximum width w2 of the smaller lumen pathway 106b is about 0.06 in. and maximum width w1 of the larger lumen pathway 106a is about 0.08 in. The septum 202 has a width w3 of about 0.02±0.002 in., while the tubes 104 have an exterior width w4 of about 0.022±0.003 in. Maximum height h2 of the smaller pathway 106a is about 0.14 in. and maximum height h1 of the larger pathway 106b is about 0.15 in.

The cut distal end 136 of the shorter tube 104b can be trimmed in a perpendicular direction or a non-perpendicular direction with respect to the longitudinal axis β. FIG. 18 shows the cut distal end 136 trimmed in a perpendicular direction with respect to axis β. Alternatively, FIG. 19 shows the cut distal end 136 trimmed in a non-perpendicular direction with respect to axis P. The non-perpendicular direction can result in any non-zero angle θ between the cut distal end 136 and the axis β. As shown in FIGS. 18 and 19, the distal extraction tip extraction portion 110b of the blood extraction tube 104b terminates proximal to the distal return tip portion 110a of the blood return tube 104a. However, also including the lumen tip segment 134 attached to the distal tip return portion 110b as shown in FIG. 18, the two distal lumen tip segments 110 have the same length, although even including the lumen tip segment 134, one or the other of the lumen tips 110 can be longer than the other.

With a distal portion of the catheter assembly 102 removed, or the tubes 104 joined so that one extends beyond the other at the distal portion 108, the lumen tip segment 134 can be joined to the catheter assembly 102 as shown in FIG. 18. The lumen tip segment 134 has been joined to the lumen tip 110b of the cut tube 104b at the cut distal end 136 such that the pathway of the cut tube 104b is in communication with the pathway of the lumen tip segment 134, thereby forming a single pathway 106b through the cut tube 104b and the lumen tip segment 134.

The lumen tip segment 134 can be attached to the catheter assembly 102 in a variety of ways. For example, the lumen tip segment 134 can be fused and/or bonded to the lumen tip 110b at the cut distal end 136. Any fusion technique and/or bonding technique can be used, such as those described above. In some embodiments, the lumen tip segment 134 can be attached in such a way as to provide a gradual transition between the luminal walls of the catheter assembly 102 and the luminal walls of the lumen tip segment 134, for instance via the insertion of a mandrel and the application of heat.

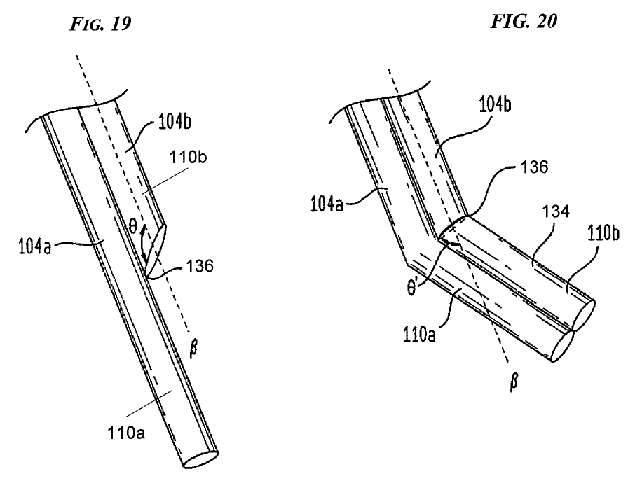

The lumen tip segment 134 can be oriented at any angle with respect to the longitudinal axis β of the cut tube 104b. Moreover, one or both of the lumen tip segment 134 and the lumen tip 110a can have a convex shape with respect to the other tip over at least some portion of its length. For example, the lumen tip segment 134 can be attached to the lumen tip 110b at an angle θ′ with respect to the axis β as shown in FIG. 18, where in this example O′ equals ninety degrees. In such a configuration, the lumen tips 110 are separate but are substantially parallel to each other. FIG. 20 shows another embodiment where the lumen tips 110 are separate and substantially parallel to each other in an angled spit tip configuration. Alternatively, as shown in FIG. 21, the lumen tip segment 134 can be oriented to the cut tube 104b at an angle θ′ less than ninety degrees. In such a configuration, the tubes 104 are separate and diverge from each other at an angle δ. When the angle θ′ is less than ninety degrees, it is typically in configurations where the cut distal end 136 has been trimmed in a non-perpendicular direction with respect to the axis β, and the angle δ is formed when the lumen tip segment 134 is joined to the cut tube 104b. However, the angle δ can be formed after the lumen tip segment 134 has been joined to the cut lumen tip 110b, e.g., by the application of heat. In another example, the design in FIG. 21 can be formed by first attaching the lumen tip segment 134 to the cut lumen tip 110b and then heating the tubes 104 to form the angle δ. Alternatively, the lumen tips 110 such as those in FIG. 21 can have an initial configuration where they are at the angle θ′ with respect to the axis P.

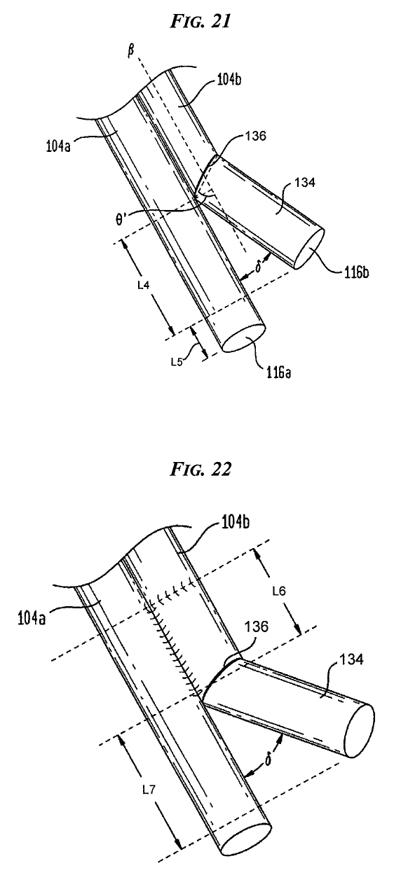

The apex of the angle δ can be located either at the junction of the cut tube 104b and the lumen tip segment 134, as shown in FIG. 21, or further toward the distal end of the catheter assembly 102. In the case that the angle δ is further toward the distal end of the catheter assembly 102, the lumen tip segment 134 can be attached (e.g., fused and/or bonded) along a length L6 of the uncut tube 104a, as shown in FIG. 22. Alternatively, the lumen tip segment 134 can be bonded to the septum along a length L6 of the uncut tube 104a and attached to the cut tube 104b at an angle θ′, as shown in FIG. 23. Typically, in these or other embodiments, the lumen tip segment 134 can also be bonded along the circumference at the junction with the cut tube 104b.

Additional information on split-tip catheters and manufacturing techniques can be found in commonly-owned, co-pending U.S. Patent Application Ser. No. 60/980,633 filed Oct. 17, 2007 entitled “Manufacture Of Split Tip Catheters,” herein incorporated by reference in it entirety.

Whether substantially parallel or diverging from one another, the lumen tips 110 of the tubes 104 are separate (at least before application of any adhesive, if any). FIG. 21 shows the tubes 104 separate for the length L4, and FIG. 22 shows the tubes 104 separate for the length L7. FIG. 21 also shows an embodiment where one of the tubes 104 is longer than the other, with the distal end 116a of the lumen tip 110a extending beyond the distal end 116b of the lumen tip segment 134 by a length L5.

Referring again to FIG. 18, the tubes 104 shown in this embodiment are substantially parallel and can be secured together with an adhesive 1600 for a length L4. Prior to the distal ends 116 of the catheter assembly 102 being inserted into a blood vessel, a full or partial portion of the lumen tips 110 of the tubes 104 can be joined to one another with the bioresorbable adhesive 1600. After insertion into the blood vessel, the bioresorbable adhesive 1600 facilitates separation of the lumen tips 110 of the tubes 104, as discussed above.

As shown in FIG. 18, the bioresorbable adhesive 1600 can be applied along a facing surface of either, or both, the lumen tips 110 of the tubes 104 to facilitate the joining of the lumen tips 110 along their longitudinal lengths prior to insertion of the distal ends 116 of the catheter assembly 102 into a blood vessel. FIG. 18 shows the bioresorbable adhesive 1600 applied along a longitudinal length L4. However, the bioresorbable adhesive 1600 need not be applied along the entire length of the facing surfaces of each tube 104 but is preferably applied such that the adhesive 1600 facilitates the joining of the lumen tips 110 of the tubes 104 prior to insertion into a blood vessel and allows the lumen tips 110 of the tubes 104 to separate after insertion. Furthermore, the bioresorbable adhesive 1600 can be applied along more than length L4 if, for example, the tubes 104 were separated for an additional length, in which case the adhesive 1600 can be applied along a length equal to L4 plus the additional length.

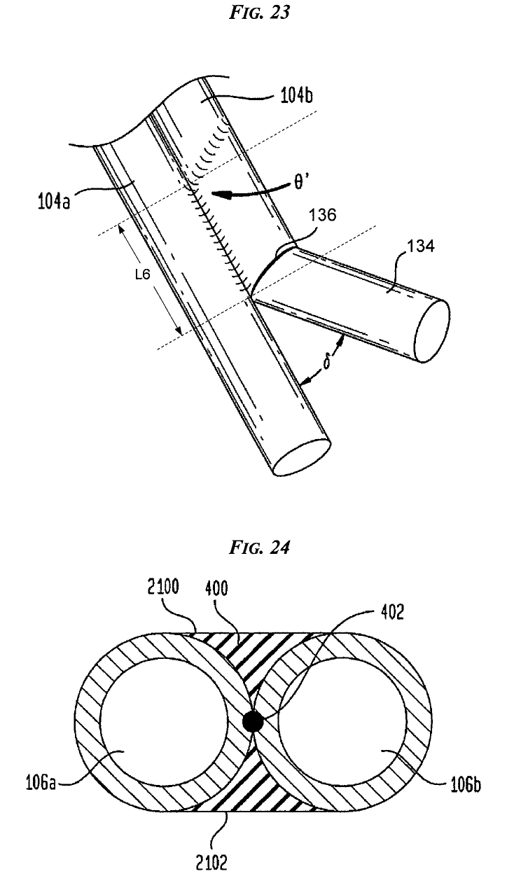

FIGS. 24-25 show cross-sections of the lumen tips 110 of the tubes 104 detailing alternate embodiments of the bioresorbable adhesive application. FIGS. 24 and 25 show bioresorbable adhesive 400 applied at a contact point 402 of the facing surfaces of the tubes 104. FIG. 24 shows one embodiment of an application of the bioresorbable adhesive 400 such that the adhesive 400, as applied, joins non-contacting surfaces 2100, 2102 of the lumen tips 110 of the tubes 104. FIG. 25 shows a variation on the embodiment shown in FIG. 24 where the bioresorbable adhesive 400 surrounds the lumen tips 110 of the tubes 104 forming a continuous cross-section of adhesive coating notwithstanding the lumen tips 110 of the tubes 104 extending therethrough. As stated above, the bioresorbable adhesive 400 need not be applied along the entire length of the lumen tips 110 of the tubes 104 but is preferably applied such that the adhesive 400 facilitates the joining of the distal extraction and return tip portions 110 of the blood extraction and blood return tubes 104 prior to insertion into a blood vessel and allows the lumen tips 110 of the tubes 104 to separate after insertion.

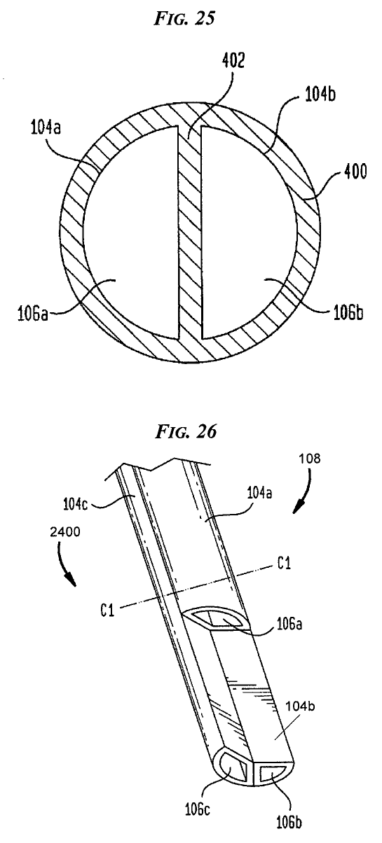

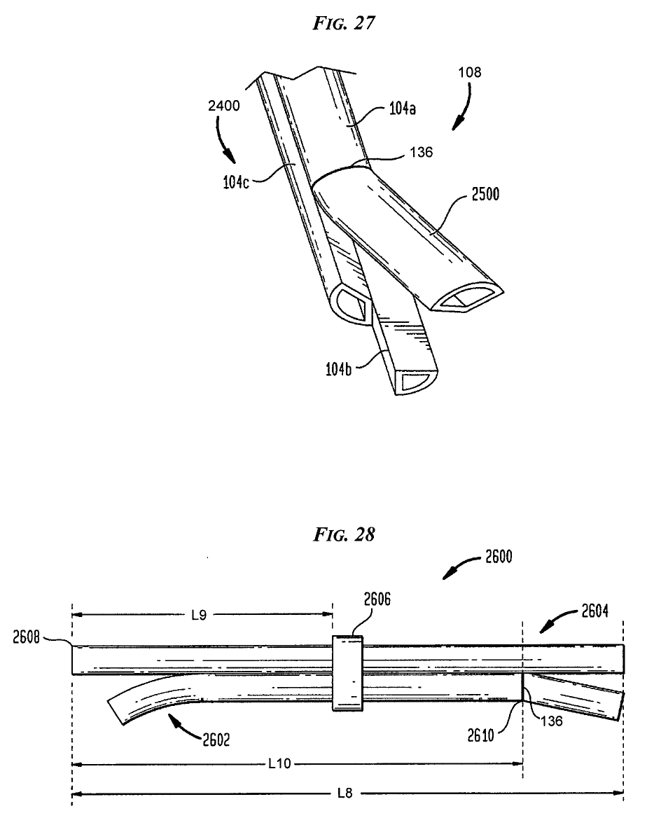

FIGS. 1-11 and 14-25 illustrate double lumen configurations, but the split tip catheter devices and methods described herein can apply to any multi-lumen configuration. For example, FIG. 26 shows an embodiment of a catheter assembly 2400 having three tubes 104a, 104b, 104c, each having respective pathways 106a, 106b, 106c. The catheter assembly 2400 can have any c1-c1 cross-sectional configuration, and in this example is shown having the one of FIG. 13. One of the tubes 104a in this example has a shorter longitudinal length at the distal portion 108 than the other tubes 104b, 104c by having an overall shorter longitudinal length, being so arranged in a staggered, step configuration when attached to the other tubes 104b, 104c, and/or being trimmed. FIG. 27 shows the catheter assembly 2400 of FIG. 26 where a second tube 104c has a shorter longitudinal length at the distal portion 108 than a longest one of the tubes 104b. A lumen tip segment 2500 has been attached to the first trimmed tube 104a, and another lumen tip segment can be attached to the second trimmed tube 104c.

For the above embodiments that describe a split distal end of a catheter, in addition to or instead of splitting the distal end, the proximal end can also be formed in a split tip configuration in any way described above with respect to the distal end (e.g., in a double split-tip or “double-Y” configuration). Such a configuration can be useful in retrograde or reverse insertions where the catheter assembly is passed through a subcutaneous tunnel from venotomy site to the remote exit location. After tunneling the catheter, fluid couplings or other attachments can be disposed to the proximal end of the lumens. FIG. 28 shows an embodiment of a catheter assembly 2600 having a split distal end 2602 and a split proximal end 2604. A hub or cuff 2606 can be attached to any location on the catheter assembly 2600 to enhance tissue ingrowth. The catheter assembly 2600 can have any dimensions, but only as an example, the catheter assembly 2600 can have a length L8 of about 38 cm, a length L9 between a distal most end 2608 of the distal end 2602 and the cuff 2606 can be about 23 cm, and a length L10 between the distal most end 2608 and a cut proximal end 2610 can be about 28 cm.

Other embodiments are within the scope of the following claims.

All publications, patent documents and other information sources identified in this application are hereby incorporated by reference.

What is claimed is:

1. A method of forming a catheter, comprising:
providing a first catheter tube having a substantially D-shaped cross-section and a second catheter tube having a substantially D-shaped cross-section; and
attaching at least a portion of longitudinal lengths of the first catheter tube and the second catheter tube along flat surfaces of the first catheter tube and the second catheter tube to form a dual lumen catheter assembly.

2. The method of claim 1, wherein the step of attaching further comprises fusing the tubes together along at least about 70% of the longitudinal length of at least one of the tubes.

3. The method of claim 1, further comprising securing a non-fused portion of the longitudinal lengths together with a bioresorbable adhesive.

4. The method of claim 1, further comprising orienting distal portions of the first catheter tube and second catheter tube such that the distal portions are separate and diverge from each other at an angle.

5. The method of claim 1, further comprising orienting distal portions of the first catheter tube and second catheter tube such that the distal portions are substantially parallel to each other.

6. The method of claim 5, further comprising orienting the distal portions such that the distal portions are separate from each other.

7. The method of claim 1, wherein the step of attaching at least a portion of longitudinal lengths further comprises heat bonding the first catheter tube and the second catheter tube.

8. The method of claim 1, wherein the step of attaching at least a portion of longitudinal lengths further comprises adhesive or chemical reaction bonding the first catheter tube and the second catheter tube.

9. The method of claim 1, further comprising orienting the first and second catheter tubes such that one tube extends longitudinally beyond the other tube.

10. The method of claim 9, further comprising removing a portion of the assembly to form a first lumen tip segment such that the first catheter tube extends longitudinally beyond the second catheter tube.

11. The method of claim 9, further comprising fusing together two tubes of different longitudinal lengths such that the first catheter tube extends longitudinally beyond the second catheter tube.

12. The method of claim 9, further comprising joining a second lumen tip segment to the second catheter tube in fluid communication with the second catheter tube.

13. The method of claim 12, wherein the second lumen tip segment has a cross-section shape different from the second catheter tube.

14. The method of claim 12, wherein the second lumen tip segment has a substantially D-shaped cross-section.

15. The method of claim 1, further comprising encasing the assembly to smoothen any irregularities along the attached portion of the longitudinal lengths.

16. The method of claim 1, further comprising forming fluid passage holes in a side of a distal portion of at least one of the catheter tubes.

17. A method of forming a catheter, comprising:
providing a first catheter tube having a cross-section including at least one substantially flat-sided surface and a second catheter tube having a cross-section including at least one substantially flat-sided surface; and
attaching at least a portion of the substantially flat-sided surfaces together to form a catheter assembly.

18. The method of claim 17, further comprising allowing a distal portion of the first catheter tube to extend beyond a distal portion of the second catheter tube when at least a portion of their substantially flat-sided surfaces are attached.

19. The method of claim 18, further comprising joining a lumen tip segment to the second catheter tube such that the lumen tip segment is in communication with the second catheter tube.

20. The method of claim 17, wherein the step of attaching at least a portion of the catheter tubes further comprises heat bonding the first catheter tube and the second catheter tube.

21. The method of claim 17, wherein the step of attaching at least a portion of the catheter tubes further comprises adhesive or chemical reaction bonding the first catheter tube and the second catheter tube.

22. The method of claim 17, further comprising encasing the catheter assembly to smoothen any irregularities along the attached surfaces.

23. The method of claim 17, wherein the first and second catheter tubes each have a substantially D-shaped cross-section.

24. A method of forming a split tip catheter, comprising:
attaching two tubes together along a portion of substantially flat surfaces of respective longitudinal lengths of the tubes; and
allowing distal portions of each of the tubes to remain unattached from each other.

25. The method of claim 24, wherein the step of attaching the two tubes together further comprises attaching the tubes along substantially planar edges of respective D-shaped cross-sections of the tubes.

26. The method of claim 24, further comprising allowing proximal portions of the tubes to remain unattached from each other.

27. The method of claim 24, further comprising orienting the tubes such that the distal portion of one tube extends longitudinally beyond the distal portion of the other tube.

28. The method of claim 27, further comprising removing at least part of the distal portion of one of the tubes to form a first lumen tip segment such that the first lumen tip segment extends longitudinally beyond the other tube.

29. The method of claim 27, further comprising joining a lumen tip segment to the tube with a shorter distal portion such that the lumen tip segment is in communication with the tube with a shorter distal portion.

30. The method of claim 27, further comprising fusing together two tubes of different longitudinal lengths such that the distal portion of one tube extends longitudinally beyond the distal portion of the other tube.

31. The method of claim 24, further comprising securing a non-attached portion of the longitudinal lengths together with a bioresorbable adhesive.

32. The method of claim 24, wherein the step of attaching two tubes together further comprises fusing the tubes together along at least about 70% of the longitudinal length of at least one of the tubes.

33. A catheter assembly, comprising:
a first catheter body having a first lumen extending longitudinally through the catheter body;
a second catheter body having a second lumen extending longitudinally through the catheter body;
the first and second catheter bodies fused together along at least about 50% of the longitudinal length of at least one of the tubes.

34. The catheter assembly of claim 33, wherein the catheter bodies are fused together along at least about 70% of the longitudinal length of at least one of the tubes.

35. The catheter assembly of claim 33, wherein the catheter bodies are fused together along at least about 80% of the longitudinal length of at least one of the tubes.

36. The catheter assembly of claim 33, wherein the catheter bodies are fused together along at least about 90% of the longitudinal length of at least one of the tubes.

37. The catheter assembly of claim 33, wherein the catheter bodies each have at least one flat surface and the bodies are fused together along their flat surfaces.

38. The catheter assembly of claim 33, wherein a non-fused portion of the longitudinal lengths are secured together with a bioresorbable adhesive.

39. The catheter assembly of claim 33, wherein a distal portion of the first catheter tube and a distal portion of the second catheter tube are separate from each other.

40. The catheter assembly of claim 33, wherein a distal portion of the first catheter tube and a distal portion of the second catheter tube are separate and diverge from each other at an angle.

41. The catheter assembly of claim 33, wherein a distal portion of the first catheter tube and a distal portion of the second catheter tube are separate and substantially parallel to each other.

42. The catheter assembly of claim 33, wherein a distal portion of the first catheter tube and a distal portion of the second catheter tube are oriented such that one tube extends longitudinally beyond the other tube.

43. The catheter assembly of claim 33, wherein the first and second lumens each comprise a substantially D-shaped cross-section.

44. The catheter assembly of claim 33, wherein one of the first and second lumens has a cross-section shape different from the other lumen along at least a portion of its longitudinal length.

45. The catheter assembly of claim 44, wherein one of the first and second lumens has a cross-section size different from the other lumen along at least a portion of its longitudinal length.

46. The catheter assembly of claim 33, wherein the assembly further comprises an outer sheath encasing the assembly along at least a portion of the fused longitudinal length.

47. The catheter assembly of claim 33, wherein the assembly further comprises fluid passage holes in a side of a distal portion of at least one of the catheter tubes.
